(19) United States
(12) Patent Application Publication
(10) Pub. No.: US 2026/0165568 A1
(43) Pub. Date: Jun. 18, 2026

(54) ENDOSCOPE AUXILIARY INFORMATION GENERATION DEVICE, ENDOSCOPE AUXILIARY INFORMATION GENERATION METHOD, ENDOSCOPE AUXILIARY INFORMATION GENERATION PROGRAM, INFERENCE MODEL TRAINING METHOD, AND ENDOSCOPE AUXILIARY SYSTEM

(71) Applicant: OLYMPUS MEDICAL SYSTEMS CORP., Tokyo (JP)

(72) Inventors: Shunsuke OKUMURA, Machida-shi (JP); Akihiro KUBOTA, Kokubunji-shi (JP); Tatsuya YUBA, Hachioji-shi (JP); Kazuo WATANABE, Hino-shi (JP); Osamu NONAKA, Machida-shi (JP)

(73) Assignee: OLYMPUS MEDICAL SYSTEMS CORP., Tokyo (JP)

(21) Appl. No.: 19/538,104

(22) Filed: Feb. 12, 2026

Related U.S. Application Data
(63) Continuation of application No. PCT/JP2023/029600, filed on Aug. 16, 2023.

(51) Int. Cl. A61B 1/04; A61B 1/00; A61B 1/05

(52) U.S. Cl. CPC A61B 1/04; A61B 1/00009; A61B 1/05

(57) ABSTRACT

An endoscope auxiliary information generation device includes an image acquisition unit configured to sequentially acquire a plurality of images of an inside of a living body obtained by an imaging unit of an endoscope that images an inside of a lumen of a subject, a region-of-attention detection unit configured to detect a region of attention having a specific feature in the image, a relative position change detection unit configured to detect a change in a relative position between the imaging unit and a target object imaged by the imaging unit on the basis of the image, and an auxiliary information generation unit configured to generate auxiliary information for guiding the endoscope to be manipulated in accordance with consecutive changes in the relative position.

### CROSS-REFERENCE TO RELATED APPLICATIONS

This application is a continuation application based on PCT Patent Application No. PCT/JP2023/029600, filed on Aug. 16, 2023, the entire content of which is hereby incorporated by reference.

### FIELD

The present disclosure relates to an endoscope auxiliary information generation device, an endoscope auxiliary information generation method, an endoscope auxiliary information generation program, an inference model training method, and an endoscope auxiliary system.

### BACKGROUND

Endoscopes have been widely used in medical and industrial fields for a long time. For example, in the medical field, a practitioner can view an endoscopic image of the inside of a subject displayed on a display device, identify a lesion, and perform treatment on the lesion using a treatment tool.

Recently, an endoscope system that extracts a region of attention such as a lesion from a medical image and notifies the practitioner of information about the extracted region of attention has been used so that overlooking a lesion by a practitioner can be prevented. For example, Japanese Unexamined Patent Application, First Publication No. 2022-103441 (hereinafter referred to as Patent Document 1) proposes an endoscope system that detects a region of attention from a medical image and changes a display aspect according to a position of the detected region of attention so that overlooking the region of attention can be prevented.

When a relative position between an imaging unit of an endoscope and a target part to be imaged is unintentionally changed due to a patient's breathing or posture change or the like, a practitioner is likely to lose sight of the region of attention. However, the conventional endoscope system described in Patent Document 1 or the like does not present a cause of the loss of sight of the region of attention or a countermeasure for the loss of sight of the region of attention.

The present disclosure has been made in consideration of such circumstances, and an objective of the present disclosure is to provide an endoscope auxiliary information generation device, an endoscope auxiliary information generation method, an endoscope auxiliary information generation program, an inference model training method, and an endoscope auxiliary system for enabling a cause or a countermeasure for the loss of sight of a region of attention such as a lesion to be presented.

### SUMMARY

According to a first aspect of the present disclosure, there is provided an endoscope auxiliary information generation device including: an image acquisition unit configured to sequentially acquire a plurality of images of an inside of a living body obtained by an imaging unit of an endoscope that images an inside of a lumen of a subject: a region-of-attention detection unit configured to detect a region of attention having a specific feature in the image; a relative position change detection unit configured to detect a change in a relative position between the imaging unit and a target object imaged by the imaging unit on the basis of the image; and an auxiliary information generation unit configured to generate auxiliary information for guiding the endoscope to be manipulated in accordance with consecutive changes in the relative position, wherein the auxiliary information generation unit generates the auxiliary information in accordance with a change in the region of attention when the relative position change detection unit determines that consecutive changes in the relative position are not changes in an image feature of a tissue in the lumen in a regular direction and detects that the change is primarily based on movement of the living body.

An endoscope auxiliary information generation device, an endoscope auxiliary information generation method, an endoscope auxiliary information generation program, an inference model training method, and an endoscope auxiliary system of the present disclosure can enable a cause or a countermeasure for the loss of sight of a region of attention such as a lesion to be presented.

### BRIEF DESCRIPTION OF DRAWINGS

FIG. 1 is a diagram showing an endoscope system according to an embodiment.

FIG. 2 is a functional block diagram of the endoscope system.

FIG. 3 is a diagram showing an inference model.

FIG. 4 is an explanatory diagram of training data.

FIG. 5 is a flowchart showing an operation of the endoscope system.

FIG. 6 is an example of information about a medical case and information about a state of a patient.

FIG. 7 is a diagram showing an example of disappearance of a region of attention when a change in a relative position is primarily based on the movement of a living body.

FIG. 8 is a diagram showing an example of disappearance of a region of attention when a change in a relative position is primarily based on the movement of the living body.

FIG. 9 is a diagram showing an example of disappearance of a region of attention when a change in a relative position is primarily based on the movement of the living body.

FIG. 10 is a diagram showing an example of disappearance of a region of attention when a change in a relative position is primarily based on the movement of the living body.

FIG. 11 is a diagram showing an example of disappearance of a region of attention when a change in a relative position is primarily based on the movement of an endoscope.

FIG. 12 is a diagram showing an example of disappearance of a region of attention when a change in a relative position is primarily based on the movement of the endoscope.

FIG. 13 is a diagram showing an example of disappearance of a region of attention when a change in a relative position is primarily based on the movement of the endoscope.

FIG. 14 is a diagram showing an example of disappearance of a region of attention when a change in a relative position is primarily based on the movement of the endoscope.

FIG. 15 is a diagram showing an example in which an image captured before the region of attention disappears is used as second auxiliary information.

FIG. 16 is a diagram showing an example in which an image captured before the region of attention disappears is used as the second auxiliary information.

FIG. 17 is an explanatory diagram of an example in which information about the patient's posture is used as the second auxiliary information.

FIG. 18 is an explanatory diagram of an example in which information about the patient's posture is used as the second auxiliary information.

FIG. 19 is a control flowchart for detecting the patient's body orientation.

FIG. 20 is an explanatory diagram of an example in which information about an orientation of a distal end portion of the endoscope is used as the second auxiliary information.

FIG. 21 is a control flowchart for displaying a history of the orientation of the distal end portion.

### DESCRIPTION

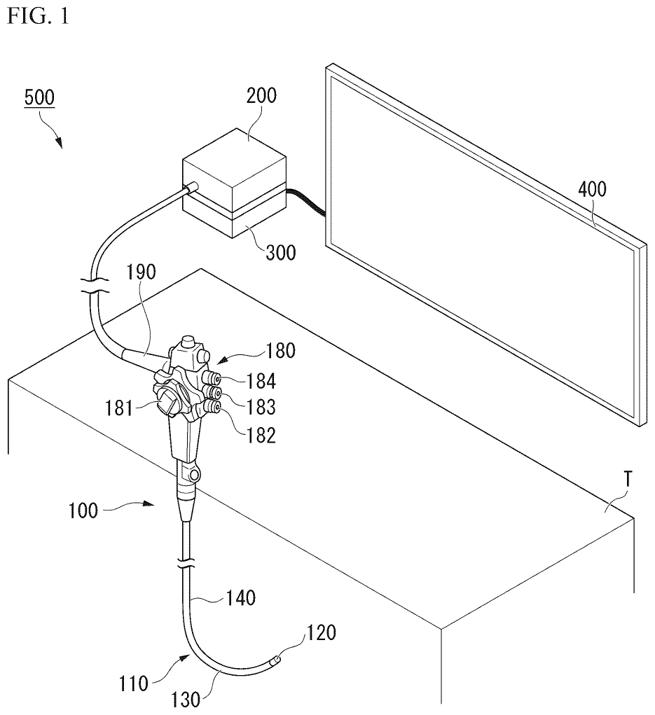

An endoscope system 500 according to an embodiment of the present disclosure will be described with reference to FIGS. 1 to 21.

### [Endoscope System 500]

FIG. 1 is a diagram showing the endoscope system 500.

The endoscope system 500 includes an endoscope 100, an image processing processor device 200, a light source device 300, and a display device 400. The image processing processor device 200 and the light source device 300 may be an integrated device (an image control device).

The light source device 300 has a light source 310 such as a light-emitting diode (LED), and controls an amount of illumination light transmitted to the endoscope 100 via the light guide 161 by controlling the light source. Thereby, observation is enabled by illuminating a lumen wall surface of a hollow organ even in a dark lumen where there is no natural light. A part at which the illumination light does not arrive appears black in a captured image D.

The display device 400 is a device that displays images generated by the image processing processor device 200, various types of information related to the endoscope system 500, and the like. The display device 400 is, for example, a liquid crystal monitor or a head-mounted display.

### [Endoscope 100]

The endoscope 100 is, for example, a device for observing and treating the inside of a patient lying on an operating table T. The endoscope 100 includes a long and thin insertion unit 110 to be inserted into the patient's body, a manipulation unit 180 connected to a proximal end of the insertion unit 110, and a universal cord 190 extended from the manipulation unit 180.

The insertion unit 110 includes a distal end portion 120, a bending portion 130 that can be bent freely, and a long and flexible tube portion 140. The distal end portion 120, the bending portion 130, and the flexible tube portion 140 are connected in that order from the distal end side. The flexible tube portion 140 is connected to the manipulation unit 180.

In addition, the image processing processor device 200 does not need to be a single device as shown in the drawing, and may be a distributed device whose functions are distributed to a plurality of devices. Some of the distributed devices may be located in different locations via a network.

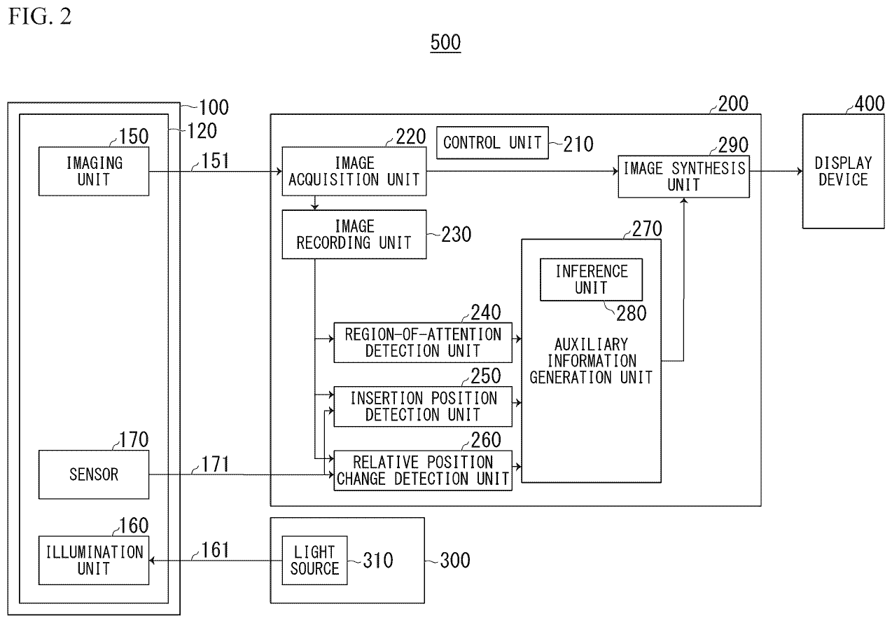

FIG. 2 is a functional block diagram of the endoscope system 500.

The distal end portion 120 has an imaging unit 150, an illumination unit 160, and a sensor 170.

The imaging unit 150 has an optical system, an imaging element configured to convert an optical signal into an electrical signal, and an analog-to-digital (AD) conversion circuit configured to convert an analog signal output by the imaging element into a digital signal. The imaging unit 150 captures an image of a subject and generates an imaging signal. The imaging signal is acquired by the image processing processor device 200 via an imaging signal cable 151.

The illumination unit 160 irradiates the subject with illumination light transmitted by the light guide 161. The light guide 161 is inserted through the insertion unit 110, the manipulation unit 180, and the universal cord 190 and connected to the light source device 300. The illumination unit 160 may have a light source such as an LED, or an optical element such as a phosphor with a wavelength conversion function.

The sensor 170 detects a position of the distal end portion 120 and a speed and direction of the distal end portion 120. The sensor 170 is, for example, an acceleration sensor, a gyro sensor, a direction sensor, a combination of these sensors, or the like. The output of the sensor 170 is acquired by the image processing processor device 200 via a signal cable 171.

The manipulation unit 180 receives a manipulation on the endoscope 100. The manipulation unit 180 has an ankle knob 181 configured to control the bending portion 130, an air/water supply button 182, a suction button 183, and a release button 184. The ankle knob 181 is a rotating handle configured to bend the bending portion 130. Manipulations input to the air/water supply button 182, the suction button 183, and the release button 184 are acquired by the image processing processor device 200. The release button 184 is a push button configured to input a manipulation to save the captured image acquired from the imaging unit 150.

The universal cord 190 connects the endoscope 100 and the image processing processor device 200. The universal cord 190 is a cable through which the imaging signal cable 151, the light guide 161, the signal cable 171, and the like pass.

### [Image Processing Processor Device 200]

As shown in FIG. 2, the image processing processor device (the endoscope auxiliary information generation device) 200 includes a control unit 210, an image acquisition unit 220, an image recording unit 230, a region-of-attention detection unit 240, an insertion position detection unit 250, a relative position change detection unit 260, an auxiliary information generation unit 270, and an image synthesis unit 290.

The image processing processor device 200 is a programmable computer that includes a processor such as a central processing unit (CPU), a memory, a recording unit, and the like. The functions of the image processing processor device 200 are implemented by the processor executing a program. At least some of the functions of the image processing processor device 200 may be implemented by a dedicated logic circuit mounted on an application-specific integrated circuit (ASIC) or a field-programmable gate array (FPGA).

The image processing processor device 200 may further include constituent elements other than a processor, a memory, and a recording unit. For example, the image processing processor device 200 may further include an image calculation unit configured to perform a part or all of image processing or an image recognition process. When the image calculation unit is further included, the image processing processor device 200 can execute the specific image processing or the image recognition processing at a high speed. The image calculation unit may be a calculator provided in a cloud server connected via the Internet.

The recording unit is a non-volatile recording medium configured to store the above-described program and data required for executing the program. The recording unit includes, for example, a writable non-volatile memory such as a flexible disk, a magneto-optical disk, a read-only memory (ROM), or a flash memory, a portable medium such as a compact disc (CD)-ROM, or a storage device such as a hard disk or a solid-state drive (SSD) built into a computer system. The recording unit may be a storage device provided in a cloud server connected via the Internet or the like.

The above-described program, for example, may be provided by a “computer-readable recording medium” such as a flash memory. The program may be transmitted from a computer that holds the program to the memory or recording unit via a transmission medium or by transmission waves in the transmission medium. The “transmission medium” for transmitting the program refers to a medium having a function of transmitting information. The medium having a function of transmitting information includes a network (a communication network) such as the Internet or a communication circuit (a communication line) such as a telephone circuit. Moreover, the above-described program may be a program for implementing some of the above-described functions. Furthermore, the above-described program may be a differential file (a differential program). The above-described functions may be implemented by a combination of the program already recorded on the computer and the differential program.

At least a part of the image processing processor device 200 may be a device separated from the image processing processor device 200. The separated device may be a calculation device provided in a cloud server connected via the Internet.

The control unit 210 controls the entire image processing processor device 200. Moreover, the control unit 210 acquires information about a medical case in which the endoscope system 500 is used (information about a type of endoscope 100 and a patient) from an in-hospital system or the like. Moreover, the control unit 210 may acquire the information about the medical case by allowing a practitioner or assistant to input the information from an input device (not shown).

The image acquisition unit 220 acquires an imaging signal from the imaging unit 150 of the endoscope 100 via the imaging signal cable 151. The image acquisition unit 220 performs imaging signal processing on the imaging signal acquired from the imaging unit 150 to sequentially acquire captured images D. The image acquisition unit 220 outputs the acquired captured images D to the image synthesis unit 290. Moreover, the image acquisition unit 220 outputs the acquired captured images D to the region-of-attention detection unit 240, the insertion position detection unit 250, the relative position change detection unit 260, and the auxiliary information generation unit 270 via the image recording unit 230.

The image recording unit 230 is a part of the recording unit described above and is a non-volatile recording medium. The image recording unit 230 is a part of the memory described above and may be a volatile recording medium. The image recording unit 230 records a plurality of captured images D that have been transmitted.

The image recording unit 230 records the plurality of captured images D (image frames and time-series images) input in chronological order. When a recording capacity of the image recording unit 230 is insufficient, the oldest captured image D is deleted. The plurality of captured images D recorded in the image recording unit 230 may be captured images D of consecutive frames, or may be captured images D in which a plurality of frames have been thinned out from consecutive frames.

The region-of-attention detection unit 240 detects a region of attention (a region of interest) from the captured image D. The region of attention may be an abnormal region such as a lesion (a first region of attention), a progress path of the insertion unit 110 of the endoscope 100 (a second region of attention), or the like. The region-of-attention detection unit 240 detects a region of attention (a region of interest) on the basis of the captured image D.

The region-of-attention detection unit 240, for example, may identify a region of attention (a region of interest) included in the captured image D by image feature determination, pattern matching, or the like. For example, the insertion position detection unit 250 compares a previously recorded image of a lesion such as a polyp with the captured image D and identifies an abnormal area (a first region of attention) included in the captured image D on the basis of a degree of similarity to a previously recorded image feature or pattern of the lesion.

The region-of-attention detection unit 240, for example, detects an abnormal area (a first region of attention) such as a lesion from the captured image D by a machine learning model for lesion detection generated by machine learning using the captured image D for learning. Moreover, the region-of-attention detection unit 240, for example, detects a path in the progress direction within the lumen (a progress path or a second region of attention) from the captured image D by a machine learning model for progress path detection generated by machine learning using the captured image D for learning.

The region-of-attention detection unit 240 transmits the detected region of attention (the detected region of interest) to the auxiliary information generation unit 270.

The insertion position detection unit 250 detects an insertion position within the lumen of the distal end portion 120 from which the captured image D is acquired. When the insertion unit 110 of the endoscope 100 is inserted into the large intestine, the insertion position detection unit 250, for example, identifies an insertion position of the captured image D by “partitioned structures (sites) within the large intestine” such as the cecum, ascending colon, transverse colon, descending colon, sigmoid colon, and rectosigmoid portion. When the insertion unit 110 of the endoscope 100 is inserted into the stomach, the insertion position detection unit 250 identifies the insertion position of the captured image D by “partitioned structures (sites) within the stomach” such as the pharynx, esophagus, and inside of the stomach.

The insertion position detection unit 250 may (B1) detect the insertion position of the captured image D on the basis of the captured image D or (B2) detect the insertion position of the captured image D on the basis of the output of the sensor 170. The detection methods (B1) and (B2) will be described below.
(B1) The insertion position detection unit 250 may identify the structure of the lumen included in the captured image D by image feature determination, pattern matching, or the like. For example, the insertion position detection unit 250 compares the captured image D with images of the parts recorded in advance and identifies the structure of the lumen contained in the captured image D on the basis of the degree of similarity to each part recorded in advance. Even a lumen that appears to be a series of similar structures may have functions at each position, and a feature may differ according to the position. There are cases where deformation appears due to the influence of adjacent organs or tissues, and the structure (site) can be identified from the features. It is only necessary for the insertion position detection unit 250 to identify the structure of the lumen by detecting such features.
(B1) The insertion position detection unit 250 may infer and identify the structure (site) of the lumen included in the captured image D using an inference model. For example, the inference model is obtained by machine learning using images of the sites recorded in advance and annotations of the sites included in the images as training data. In such a method, the above-described intraluminal features are also used for inference.
(B2) The insertion position detection unit 250 may determine the insertion position from (the accumulation of) information obtained from the output of the sensor 170 (the acceleration, speed, direction, posture, and the like of the distal end portion 120) to identify the insertion position. Likewise, the insertion position detection unit 250 may determine a degree of progress in the lumen according to a change over time in the captured image D and accumulate the change over time to detect the insertion position.

The insertion position detection unit 250 may identify the insertion position by combining the detection methods (B1) and (B2).

The insertion position detection unit 250 transmits the insertion position in the lumen of the distal end portion 120 from which the captured image D is acquired (the partitioned structure within the lumen) to the auxiliary information generation unit 270.

The relative position change detection unit 260 detects a change in a relative position between the distal end portion 120 of the endoscope 100 and a target part to be imaged. The relative position change detection unit 260 can determine whether the change in the relative position is primarily based on the movement of the endoscope 100, whether the change in the relative position is primarily based on the movement of the living body, or whether the change in the relative position is based on both movements.

The relative position change detection unit 260 may (C1) detect the change in the relative position on the basis of the captured image D or (C2) detect the change in the relative position on the basis of the output of the sensor 170. The detection methods (C1) to (C2) will be described below.

(C1) The relative position change detection unit 260 may detect the change in the relative position according to image recognition of the captured image D. Specifically, when the endoscope 100 (the distal end portion 120) itself is moving, there are features in which the image features such as the unevenness of the intraluminal tissue and blood vessels detected by the imaging unit 150 change in an approximately regular direction (in a direction radially spreading from the approximate center of the screen to the outside of the screen, in the opposite direction at the time of insertion or removal, or in a bending direction at the time of bending) and things previously invisible from the other side of the moving lumen direction (the shadow of the lumen) become visible. Therefore, when it is determined that the image features of the intraluminal tissue have changed in an approximately regular direction between image frames obtained in chronological order, the relative position change detection unit 260 mainly determines that the endoscope 100 has moved. The approximately regular direction may be a direction identical to any one of the up, down, left, and right directions of the screen, or the same direction radially as inserted or removed. In addition, these may occur in combination. It is not necessary to determine this image movement for all pixels or all image parts and it is only necessary to determine representative pixels or image parts.

On the other hand, if the endoscope 100 (the distal end portion 120) itself is almost stationary but the luminal tissue moves, when the image frames obtained in chronological order are compared, the overall image pattern including the obtained image features does not move in a regular direction. Therefore, when it is determined that the image features of the luminal tissue have not changed in a regular direction, it is determined that the endoscope (the distal end portion) itself is almost stationary and that mainly the luminal tissue has moved. Moreover, when the lumen is viewed in a longitudinal direction of the lumen and the other side contracts, the image feature changes in such a way that the tissue in a specific direction (or the color in a specific direction) appears to cover over it. When the position of a polyp or the like changes up and down, if no deformation is confirmed at a site where the polyp is formed, it can be determined that the imaging unit 150 at the distal end of the endoscope is shaking up and down.

In such a detection method, the relative position change detection unit 260 may determine a change in the obtained image for each image frame obtained in chronological order, determine whether it is advancing and retracting along the lumen, and convert a speed into a position by determining the speed from the change and or determine the position from image information inside the lumen. Moreover, if the orientation of the imaging unit 150 of the endoscope 100 or the like is similarly determined from image information within the lumen, the relative position change detection unit 260 can determine in which direction the endoscope's distal end (or the imaging unit 150) is facing at which position of which organ site.

Also, when what has been contracted expands, it results in an image pattern change in which the tissue in a specific direction (the color in a specific direction) retracts radially from the surroundings, and something (for example, the shadow of a lumen in the hollow organ) appears from the other side. When the position of a polyp or the like changes up and down, if deformation of the site where the polyp is formed is confirmed at the same time, it is possible to determine that the target object is moving without determining that the imaging unit at the distal end of the endoscope is shaking.

Moreover, when both the endoscope 100 (the distal end portion 120) and the luminal tissue move relatively, because these features occur in a complex manner, it is only necessary for the relative position change detection unit 260 to identify and detect these features.
(C1) The relative position change detection unit 260 may infer a change in relative position using an inference model. For example, the inference model is obtained by machine learning using a result of annotating what type of relative position change it is for a plurality of image frames as training data.
(C2) The relative position change detection unit 260 may detect the change in relative position using an output of the sensor 170 (the acceleration, speed, direction, posture, and the like of the distal end portion 120). When acceleration has occurred in the distal end portion 120, the relative position change detection unit 260 can calculate a change in the speed or position using time information. The relative position change detection unit 260 can determine that a change in a posture or a change in a direction is a change in a direction in which the distal end portion 120 is facing.

The relative position change detection unit 260 may detect the change in the relative position between the distal end portion 120 of the endoscope 100 and the target part of the imaging target by combining the detection methods (C1) to (C2).

The relative position change detection unit 260 transmits the detected change in the relative position to the auxiliary information generation unit 270.

The auxiliary information generation unit 270 generates auxiliary information for guiding the endoscope 100 to be manipulated in accordance with consecutive changes in a relative position between the distal end portion 120 of the endoscope 100 and a target part of an imaging target. Specifically, the auxiliary information generation unit 270 generates auxiliary information for guiding the endoscope 100 to be manipulated on the basis of detection results of the region-of-attention detection unit 240, the insertion position detection unit 250, and the relative position change detection unit 260. The auxiliary information generation unit 270 may generate the auxiliary information on a rule basis or may generate the auxiliary information using an inference model 281 held by the inference unit 280.

Here, the “auxiliary information” includes the cause of the disappearance of the region of attention in the plurality of captured images D (image frames and time-series images) input to the image recording unit 230 in chronological order and a countermeasure to recover from the disappearance. In addition, the auxiliary information may include at least one of the cause of the disappearance of the region of attention and the countermeasure to recover from the disappearance.

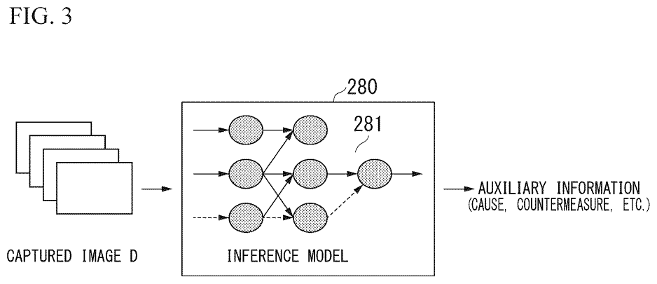

FIG. 3 is a diagram showing the inference model 281.

The inference model 281 is a model that identifies image frames before and after disappearance of the region of attention from a plurality of image frames (captured images for learning) and is trained using training data including annotations related to a cause of the disappearance and a countermeasure for the image frame after the disappearance of the region of attention. The inference model 281 is, for example, a neural network, and is trained by deep learning. The inference model 281 is not limited to a neural network, and may be another machine learning model that can output information for an input image.

The input of the inference model 281 is a plurality of captured images D (image frames and time-series images) that are input in chronological order. The input of the inference model 281 may include the output of the sensor 170. The output of the inference model 281 is auxiliary information.

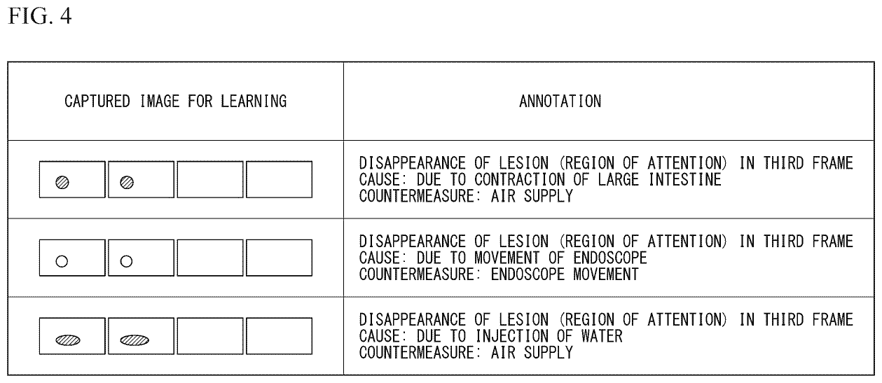

FIG. 4 is an explanatory diagram of the training data.

A plurality of image frames (a sequence of still images) obtained in endoscopic examinations of a plurality of medical cases are used as training data. The training data is a combination of a plurality of image frames (captured images for learning) and annotations configured to identify the image frames before and after the disappearance of the region of attention from the plurality of image frames and related to the cause of the disappearance and the countermeasure for the image frames after the disappearance of the region of attention. The inference model 281 is a model trained to output corresponding annotations for the input image frames (captured images for learning) using the training data.

As shown in FIG. 4, the training data desirably includes training data in which the region of attention has disappeared due to the movement of the target object, training data in which the region of attention has disappeared due to the movement of the endoscope 100, and training data in which the region of attention has disappeared due to a change in the target object due to water supply (irrigation), air supply, or the like. The inference model 281 trained using such training data (image changes in which causes and countermeasures are annotated) can infer various causes and various countermeasures for the disappearance of the region of attention. To create such training data, the images may be recorded in the image recording unit 230 of FIG. 2 or may be recorded in a recording unit (not shown). A database associated with information (meanings) such as what type of features the image has and what type of annotations the image has may also be recorded. Because such meanings for each image may be equivalent to medical knowledge, they may be referred to as a knowledge database. The knowledge database may include information such as what image features an image of which position in the lumen of which organ has. Moreover, an inference model learning unit that uses such information to create the inference model 281 is not shown in FIG. 2, but may be considered as an important element of the present embodiment.

In other words, the present embodiment is characterized by providing an inference model learning method for detecting a region of attention having a specific feature in an in-image region from consecutive images obtained by the image acquisition unit 220 using the endoscope 100 for observing the inside of a living body, identifying image frames before and after the disappearance of the above-described specific feature from the images obtained by the image acquisition unit 220, annotating a cause of the disappearance and a countermeasure for the frame after the disappearance to create training data, and inferring and outputting the cause of the disappearance and the countermeasure when an image in which the region of attention having the specific feature has disappeared from an endoscopic image is input.

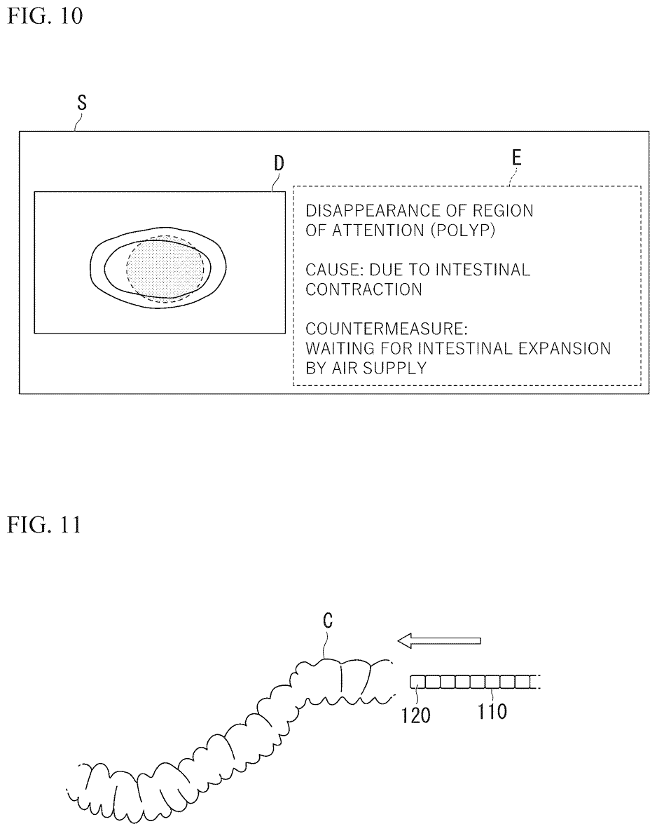

The image synthesis unit 290 generates a synthetic image S including a captured image D and auxiliary information E generated by the auxiliary information generation unit 270 (see FIG. 10). The image synthesis unit 290 sequentially creates synthetic images S corresponding to the captured images D sequentially generated by the image acquisition unit 220, and outputs the synthetic images S to the display device 400. The display device 400 sequentially displays the received synthetic images S.

### [Manipulation of Endoscope System 500]

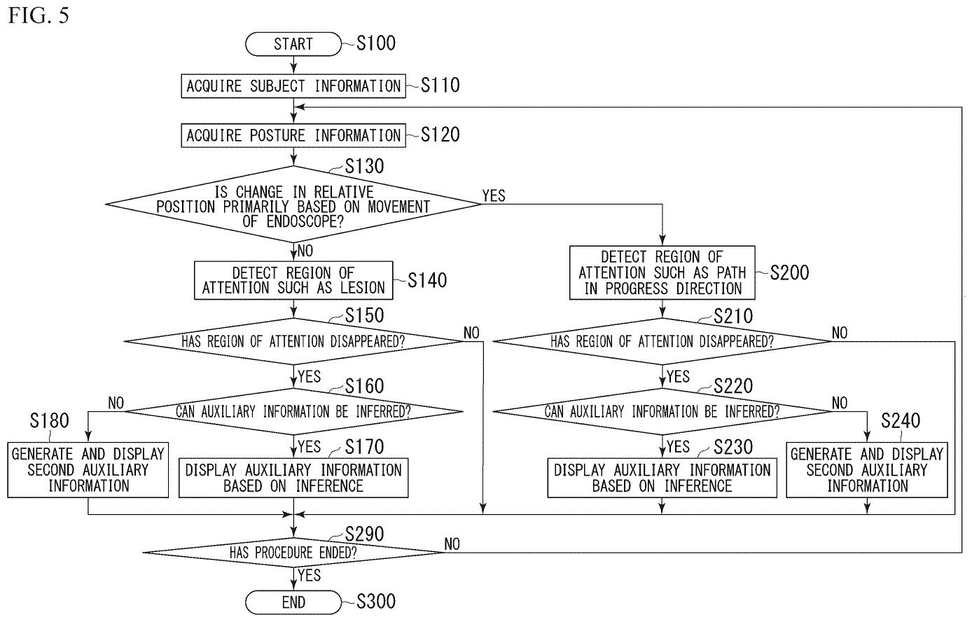

Next, the operation of the endoscope system 500 (an auxiliary information generation method) will be described. Specifically, a procedure for observing and treating the lumen wall inside the large intestine using the endoscope system 500 will be described. Hereinafter, the description will be given along the flowchart showing the operation of the endoscope system 500 shown in FIG. 5.

### <Step S110>

In step S110, the control unit 210 acquires information about a medical case in which the endoscope system 500 (information about a type of endoscope 100 and a patient) is used from an in-hospital system or the like. The control unit 210 may acquire information about the medical case by allowing the practitioner or assistant to input the information from an input device (not shown).

### <Step S120>

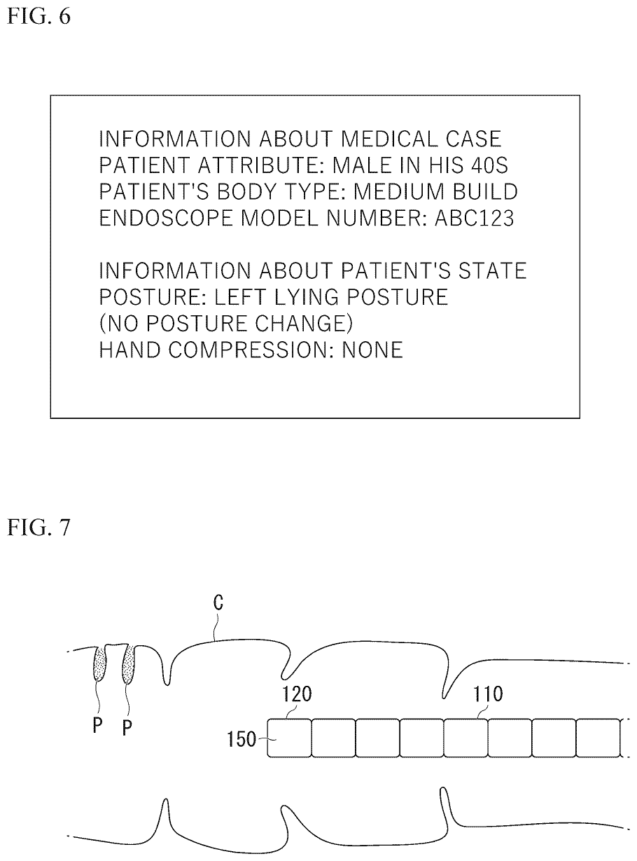

In step S120, the control unit 210 acquires information about a state of the patient (including posture information). The control unit 210 may acquire the information about the state of the patient by allowing the practitioner or assistant to input the information from an input device (not shown). FIG. 6 shows an example of information about the medical case and information about the state of the patient. The information about the state of the patient is re-input each time the state of the patient is updated. A “left lying posture,” which is a posture exemplified in FIG. 6, is a posture in which saliva is less likely to be aspirated compared to lying on the back and water tends to accumulate in the large bay of the stomach, making reflux from the stomach less likely to occur.

### <Step S130>

In step S130, the relative position change detection unit 260 detects whether there is a change in a relative position between the distal end portion 120 of the endoscope 100 and a target part of an imaging target. When there is a change in the relative position, the relative position change detection unit 260 determines whether the change in the relative position is primarily based on the movement of the endoscope 100 or whether the change in the relative position is primarily based on the movement of the living body. When the change in the relative position is primarily based on the movement of the endoscope 100, the endoscope system 500 subsequently executes step S200. When the change in the relative position is primarily based on the movement of the living body, the endoscope system 500 subsequently executes step S140.

When the change in the relative position is based on both the movement of the endoscope and the movement of the living body, the endoscope system 500 may perform both the processing of step S140 and subsequent steps and the processing of step S200 and subsequent steps.

### <Step S140>

In step S140, the region-of-attention detection unit 240 detects a region of attention (a first region of attention) such as a lesion. The endoscope system 500 subsequently executes step S150.

### <Step S150>

In step S150, the region-of-attention detection unit 240 determines whether the region of attention has disappeared. When the region of attention has disappeared, the endoscope system 500 subsequently executes step S160. When the region of attention has not disappeared, the endoscope system 500 subsequently executes step S290.

### <Step S160>

In step S160, the auxiliary information generation unit 270 determines whether auxiliary information (the cause and the countermeasures) for the disappeared region of attention can be inferred by the inference model 281. When the auxiliary information can be inferred, the endoscope system 500 subsequently executes step S170. When the auxiliary information cannot be inferred, the endoscope system 500 subsequently executes step S180.

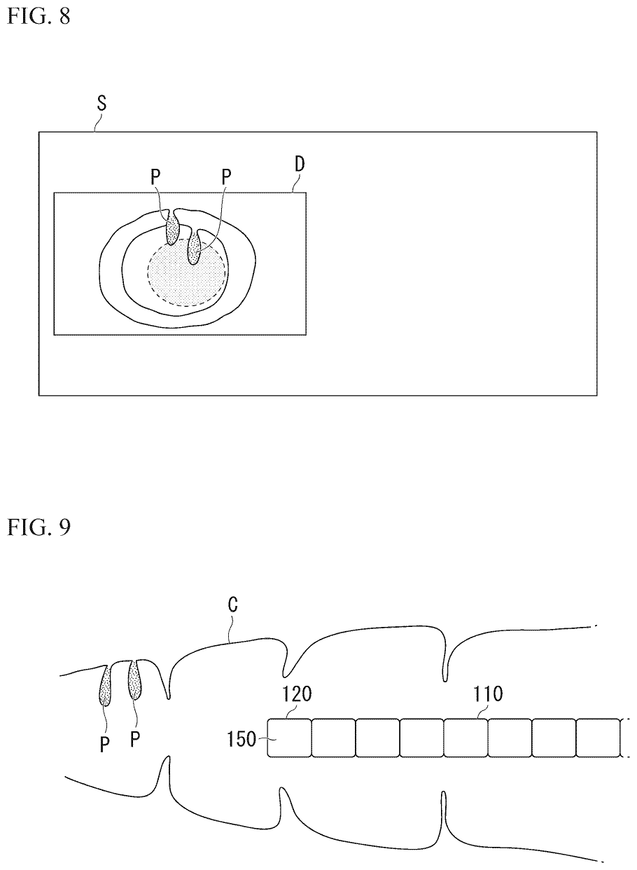

FIGS. 7 to 10 are diagrams showing an example of disappearance of a region of attention when the change in the relative position is primarily based on the movement of the living body. The imaging unit 150 provided on the distal end portion 120 of the endoscope 100 shown in FIG. 7 is arranged at a position where a polyp P can be imaged in a large intestine C. FIG. 8 is a synthetic image S including a captured image D captured by the imaging unit 150 shown in FIG. 7. The polyp P can be observed in the captured image D shown in FIG. 8. As shown in FIG. 9, the large intestine C, which is a living body, contracts (non-rigid body movement), and the imaging unit 150 cannot observe the polyp P that was being observed in the large intestine C. In this case, as shown in FIG. 10, the polyp P (the region of attention) disappears in the captured image D. The auxiliary information generation unit 270 infers auxiliary information E as shown in FIG. 10 by using the inference model 281.

### <Step S170>

In step S170, the image synthesis unit 290 generates a synthetic image S including a captured image D and auxiliary information E generated by the auxiliary information generation unit 270 as shown in FIG. 10 and outputs the synthetic image S to the display device 400. The display device 400 displays the received synthetic image S.

The endoscope system 500 subsequently executes step S290.

### <Step S180>

In step S180, the auxiliary information generation unit 270 generates and displays second auxiliary information. Details of the second auxiliary information generated in step S180 will be described below. The endoscope system 500 subsequently executes step S290.

### <Step S200>

In step S200, the region-of-attention detection unit 240 detects a path in a progress direction within the lumen (a progress path and a second region of attention). In addition, in step S200, the region-of-attention detection unit 240 may detect a region of attention (a first region of attention) such as a lesion. The endoscope system 500 subsequently executes step S210.

### <Step S210>

In step S210, the region-of-attention detection unit 240 determines whether the region of attention has disappeared. When the region of attention has disappeared, the endoscope system 500 subsequently executes step S220. When the region of attention has not disappeared, the endoscope system 500 subsequently executes step S290.

### <Step S220>

In step S220, the auxiliary information generation unit 270 determines whether auxiliary information (the cause and the countermeasures) for the disappeared region of attention can be inferred by the inference model 281. When the auxiliary information can be inferred, the endoscope system 500 subsequently executes step S230. When the auxiliary information cannot be inferred, the endoscope system 500 subsequently executes step S240.

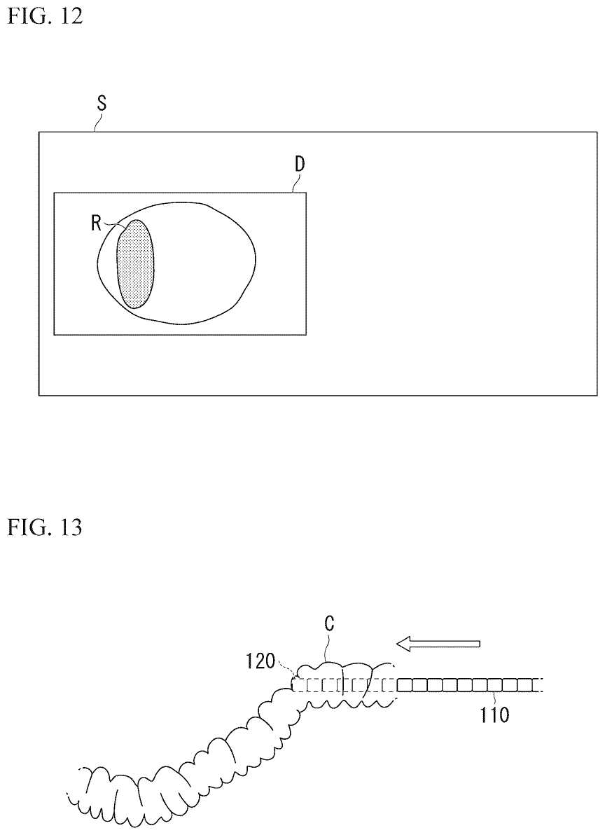

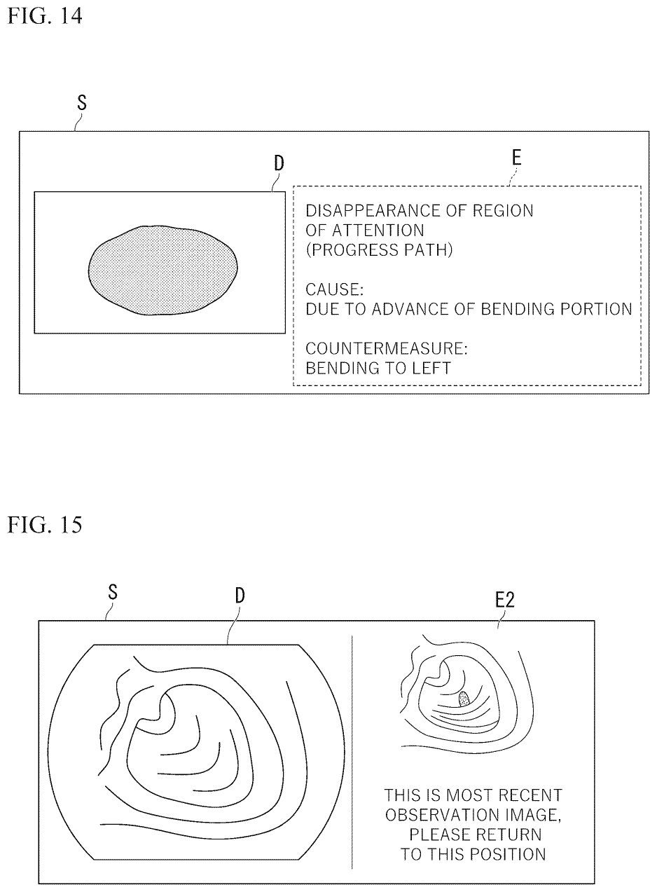

FIGS. 11 to 14 are diagrams showing an example of the disappearance of the region of attention when the change in the relative position is primarily based on the movement of the endoscope 100. As shown in FIG. 11, a shape of a lumen such as the large intestine C is complex and varies from person to person, making it difficult for the practitioner to determine a path of a progress direction (a progress path). Furthermore, it is difficult to observe the up, down, left, and right directions of the captured image D in association with the up, down, left, and right directions in the progress direction of the lumen. Before the distal end portion 120 of the insertion unit 110 approaches the bent part of the large intestine C as shown in FIG. 11, it is easy to observe a path R of the progress direction as in the captured image D shown in FIG. 12. However, as shown in FIG. 13, when the distal end portion 120 of the insertion unit 110 approaches the bent part of the large intestine C, the path R of the progress direction (the region of attention) disappears as in the captured image D shown in FIG. 14 due to the distal end portion 120 approaching the lumen wall. The auxiliary information generation unit 270 infers auxiliary information E as exemplified in FIG. 14 using the inference model 281.

### <Step S230>

In step S230, the image synthesis unit 290 generates a synthetic image S including the captured image D and the auxiliary information E generated by the auxiliary information generation unit 270, as shown in FIG. 14, and outputs the synthetic image S to the display device 400. The display device 400 displays the received synthetic image S. The endoscope system 500 subsequently executes step S290.

### <Step S240>

In step S240, the auxiliary information generation unit 270 generates and displays second auxiliary information. Details of the second auxiliary information generated in step S180 will be described below. The endoscope system 500 subsequently executes step S290.

### <Step S290>

In step S290, the control unit 210 determines whether the procedure has ended. When it is determined that the procedure has not ended, the control unit 210 executes the processing from steps S120. When it is determined that the procedure has ended, the control unit 210 executes step S300 and ends the control flow shown in FIG. 5.

If there is a region-of-attention detection function of detecting a region of attention having a specific feature in an intraluminal image during an intraluminal examination, the image features will change even if a manipulation for inserting or bending the endoscope 100 is performed, but this change can be detected. This change in the image feature is a change in the relative position between the imaging unit 150 and the target object imaged by the imaging unit 150. Specifically, when the region of attention is detected, it is possible to determine a situation at that time (the position of the distal end of the endoscope and the situation of the lumen in the progress direction) on the basis of an image part having a specific feature or to determine a manipulation on the endoscope 100 or the movement of the lumen in consecutive changes in the image feature and the like on the basis of an image part having a specific feature. Therefore, it is possible to generate endoscope auxiliary information in accordance with consecutive changes in the relative position by providing a relative position change detection function of detecting the changes.

For example, when the center of the captured image D is dark and the insertion operation of the insertion unit 110 can proceed straight, if the bending portion 130 is to be bent, auxiliary information (such as “OK to proceed straight”) can be generated to guide the user not to bend the bending portion 130. Moreover, when proceeding straight would result in hitting the inner wall of the lumen, auxiliary information (such as “Don't proceed straight”) can be generated to guide the user to prevent a collision caused by proceeding straight. Moreover, if an image feature of a hole in the lumen is detected on the right within the screen, auxiliary information can be generated to guide the user to bend the bending portion 130 to the right. Such information is auxiliary information for supporting the path of the progress direction within the living body. The auxiliary information may utilize an inference result of the inference model 281 to which the captured image D is input.

### [Generation of Second Auxiliary Information]

Even if the auxiliary information cannot be inferred by the inference model 281, the auxiliary information generation unit 270 generates the second auxiliary information according to a rule base in steps S180 and S240. Three types of second auxiliary information E2 will be described below.

### < (1) Second Auxiliary Information: Use of Image Captured Before Region of Attention Disappears>

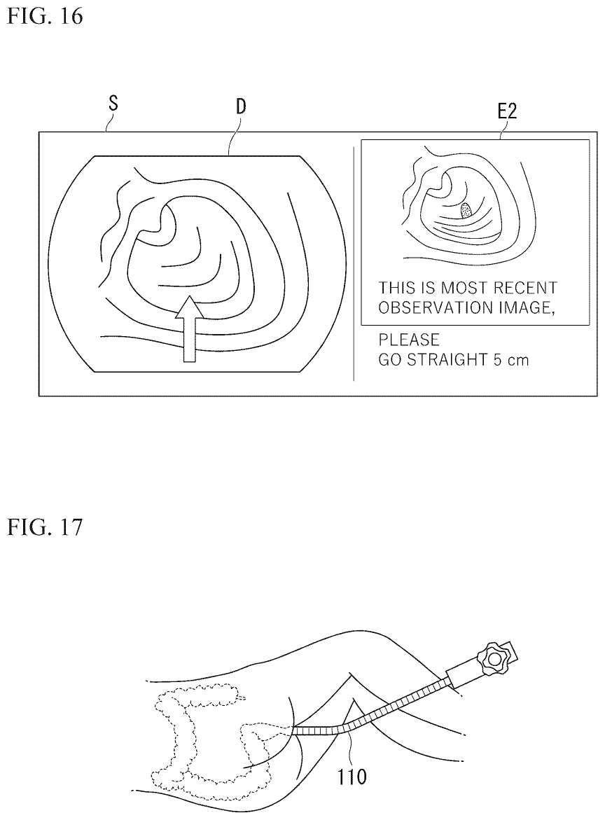

FIGS. 15 and 16 are diagrams showing an example in which the captured image D before the region of attention disappears is used as the second auxiliary information E2. The second auxiliary information E2 shown in FIG. 15 is displayed as an image that is the captured image D before the region of attention disappears and presents the practitioner with a countermeasure to return the imaging unit 150 of the endoscope 100 to the position where the image was captured. The second auxiliary information E2 shown in FIG. 16 further presents a direction and distance to move the imaging unit 150 of the endoscope 100 as the countermeasure.

< (2) Second Auxiliary Information: Use of Information about Patient's Posture>

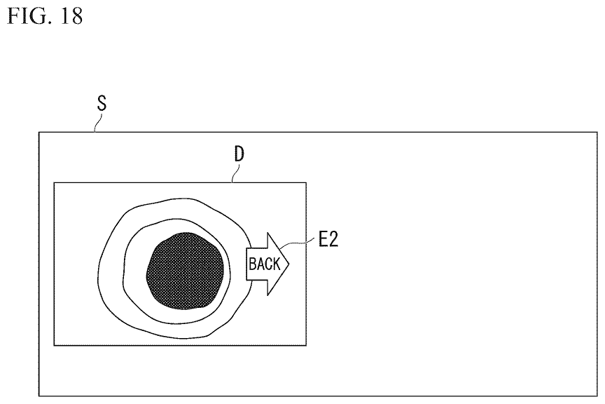

FIGS. 17 and 18 are diagrams showing an example in which information about the patient's posture is used as the second auxiliary information E2. As is clear from FIG. 17, relative positions of organs and a direction in which the lumen of each organ progresses (hereinafter also referred to as a “progress direction”) are determined to some extent by anatomical features of a human body. The progress direction may sag due to the influence of gravity, but the influence of sagging may be corrected. Moreover, when the distal end of the endoscope is facing in a direction in which the lumen progresses, a black circular image feature indicating the hole in the lumen is detected in the center within the image. A relationship between the direction of gravity (and the horizontal direction) and the progress direction of the lumen at each part obtained according to an anatomical organ arrangement feature by the patient's posture can be known. When there is an image feature of the lumen hole in the center of the captured image D obtained by the imaging unit 150, it can be known that the distal end of the endoscope is facing in the progress direction of the lumen. If the direction of gravity in the captured image D is already known and an acceleration sensor or the like is built into the distal end portion 120, it is possible to determine whether the distal end portion 120 is looking up or down in the direction of gravity by supplementing this information. Such anatomical features (information about a positional relationship of organs in the human body and a change in an orientation corresponding to the organ position in the lumen direction) may be recorded in the recording unit 230 of FIG. 2 or another recording unit (not shown), and may be made available for reference as a knowledge database (a database in which medical knowledge is digitized, recorded, and made available for reference).

A direction in which the bending portion 130 is bent when the insertion unit 110 of the endoscope 100 passes through a structure (a site) of a lumen (e.g., the large intestine) varies with the patient's posture. For example, when the patient is in the “left lying posture” as shown in FIG. 17 and the distal end portion 120 is inserted into the rectum so that the top and bottom of the distal end portion 120 (the top and bottom of the captured image D obtained from the imaging unit 150) match the top and bottom of the direction of gravity, the left side of the captured image D is the patient's back side in the above-described anatomical progress direction of the lumen.

When the endoscope 100 is inserted, auxiliary information for supporting the path of the progress direction can help the practitioner. The auxiliary information is generated according to information for identifying the above-described lumen (e.g., the large intestine), the patient's posture at the time of insertion (a direction in which the rectum faces relative to the direction of gravity) using the up-down direction of the captured screen D as a reference when the endoscope is inserted, and anatomical information about a direction of lumen bending that occurs with the insertion into the specific lumen. For example, if the rectum is aligned in the horizontal direction, unless the endoscope 100 is twisted, the bending portion 130 must be bent to the left (anatomically toward the back, but the relationship between the top and bottom changes with the posture) and then bent downward to pass through the sigmoid colon.

On the other hand, if the posture of the patient is in the “right lying posture” and the distal end portion 120 is inserted into the rectum so that the top and bottom of the distal end portion 120 match the top and bottom of the direction of gravity, the right side of the captured image D is the patient's backside based on the above-described anatomical progress direction of the lumen.

If the distal end portion 120 is further inserted from the rectum through the sigmoid colon, because the progress direction of the lumen is switched by the anatomical features of the rectum and sigmoid colon, the insertion direction of the distal end of the endoscope changes. For example, if the rectum is aligned in the horizontal direction, unless the endoscope 100 is twisted, the bending portion 130 must be bent to the right (anatomically toward the back, but the relationship between the top and bottom changes with the posture) and then bent upward to pass through the sigmoid colon.

In this way, when auxiliary information for supporting the path of the progress direction of the endoscope 100 is displayed by inserting the endoscope, the auxiliary information is generated according to information for identifying which organ the lumen is, and anatomical information about a direction of lumen bending that occurs with the above-described insertion into the specific lumen on the basis of the up-down direction of the captured screen D when the endoscope is inserted and the examination posture of the subject described above.

In addition, the distal end portion 120 is inserted into the lumen so that the top and bottom of the distal end portion 120 (the top and bottom of the captured screen D) match the top and bottom of the direction of gravity, and the up-down direction does not change if the endoscope 100 simply proceeds straight without being twisted. However, it should be noted that when the bending portion 130 is bent, the relationship between the top and bottom of the captured screen D and the top and bottom of the direction of gravity may change. However, if the direction in which the bending portion 130 is bent is known in advance, the relationship between the top and bottom of the captured screen D and the top and bottom of the direction of gravity after the bending portion 130 is bent can be determined. Moreover, if there is a twist during insertion, because the feature will appear in the captured image D during the twisting process, auxiliary information corrected in consideration of the twist may be generated. However, before the bending portion 130 is bent and the distal end of the endoscope progresses into a lumen extending in a different direction, it is not easy to bend and insert the distal end of the endoscope toward the sigmoid colon via the rectum in the first place. The practitioner must ascertain a direction in which the bending portion 130 is bent in consideration of the patient's posture. Therefore, in the present embodiment, the practitioner is supported by illustrated guides such as arrows. As shown in FIG. 18, the second auxiliary information E2 desirably includes the patient's body orientation. If the patient's body orientation in the captured image D is known, the practitioner can roughly ascertain the direction in which the bending portion 130 is bent according to information about the lumen bending direction based on the top and bottom relationship of the screen during insertion or according to anatomical changes in the lumen direction and gravity direction information. The second auxiliary information E2 shown in FIG. 18 indicates the orientation of the patient's back side in the captured image D by the direction of an arrow. Even if there is no information about the direction of the patient's body, it is possible to automatically determine the direction of the patient's body from the features of the captured image D at the time of insertion and the like.

Here, the reason why the practitioner needs to ascertain left and right in relation to the top and bottom of the captured image D and the top and bottom of the direction of gravity is that the manipulation unit 180 has a manipulation method for bending the distal end of the endoscope in up, down, left, and right directions. The essence of generating auxiliary information is to generate auxiliary information for allowing the practitioner to correctly manipulate the manipulation unit 180 by looking at the up, down, left, and right arrow displays. Therefore, the auxiliary information may be a manipulation instruction indicating an appropriate manipulation to the manipulation unit 180 in addition to a display indicating up, down, left, and right directions. The manipulation instruction may be a sound or a vibration.

A direction in which the lumen bends changes as a site progresses deeper into the lumen, for example, from the rectum to the sigmoid colon or from the sigmoid colon to the descending colon, but it is almost anatomically determined. Therefore, it is necessary to determine a position of the distal end portion 120 of the endoscope 100 within the lumen of a specific organ and a situation in which the distal end portion 120 cannot progress deeper into the lumen unless it bends there. These can be determined from changes in the images and image features by sequentially acquiring a plurality of captured images D of the inside of the living body obtained by the imaging unit 150 of the endoscope 100.

For example, a site where the distal end of the endoscope should progress straight appears darkened because the center of the screen is in a state in which light from the light source radiating the light is not reflected back for the imaging unit 150. Because a lumen is generally cylindrical, a round and dark image is obtained. However, at the point where the lumen bends, the front appears to be a wall. In this way, the region-of-attention detection unit 240 is required to detect the region of attention having the specific feature in the endoscopic image. The region of attention in this image changes in a manipulation of the insertion or bending of the endoscope 100, and the image features also change. This change in the image feature (this change in the region of attention) is a change in the relative position between the imaging unit 150 and the target object imaged by the imaging unit 150. The region is defined as a region of attention because it is determined based on an image part having a specific feature. Therefore, by providing a function for detecting a change in the relative position to detect this, endoscope auxiliary information can be generated in accordance with consecutive changes in the relative position.

For example, when the center of the captured image D is dark and the insertion operation of the insertion unit 110 can proceed straight, if the bending portion 130 is to be bent, auxiliary information (such as “OK to proceed straight”) can be generated to notify the user that the bending portion 130 does not need to be bent. Moreover, when proceeding straight would result in hitting the inner wall of the lumen, auxiliary information (such as “Don't proceed”) can be generated to guide the user to prevent a collision caused by proceeding straight. Moreover, if an image feature of a hole in the lumen is detected on the right side of the screen, auxiliary information can be generated to guide the user to bend the bending portion 130 to the right. The auxiliary information may use the inference result of the inference model 281 to which the captured image D is input. Moreover, even if such image features that serve as a hint cannot be obtained, the auxiliary information generation unit can generate auxiliary information by combining the information about the direction of the lumen bending based on the anatomical knowledge described above.

In addition, in the present embodiment, the auxiliary information is information about a countermeasure for the loss of sight of the region of attention (for example, displaying the progress direction of the lumen), but the auxiliary information is not limited thereto. The auxiliary information may be information about a cause of the loss of sight of the region of attention. For example, according to the position inside the lumen, the practitioner may also be supported by auxiliary information such as “The lumen is curved, so please look for a hole in the lumen.”

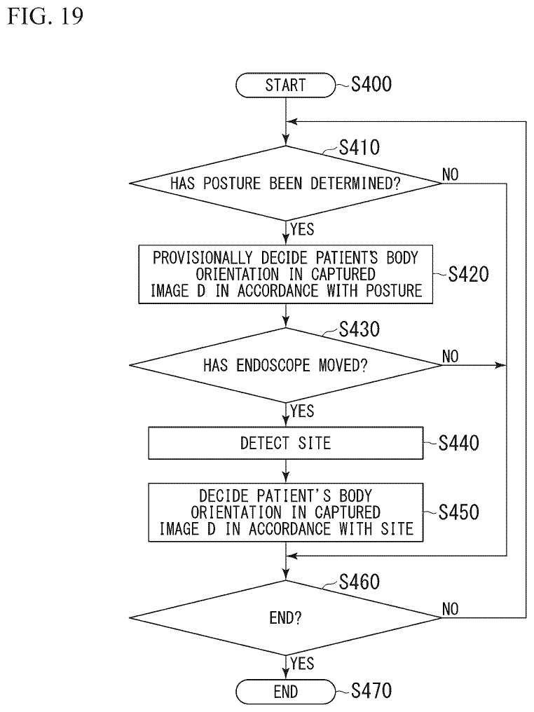

FIG. 19 is a control flowchart for detecting the patient's body orientation.

In step S410, the control unit 210 determines the patient's posture on the basis of input information about the patient's state. In step S420, the auxiliary information generation unit 270 provisionally decides the patient's body orientation in a captured image D according to the patient's posture. In step S430, the auxiliary information generation unit 270 determines whether the distal end portion 120 of the endoscope 100 has been moved on the basis of the captured image D or the output of the sensor 170. When the distal end portion 120 has been moved, the insertion position detection unit 250 detects an insertion position into which the distal end portion 120 of the endoscope 100 has been inserted in step S440. In step S450, the auxiliary information generation unit 270 decides the orientation of the patient's body in the captured image D according to the detected insertion position (site) of the distal end portion 120 in consideration of the patient's posture.

### < (3) Second Auxiliary Information: Use of Information Related to Orientation of Distal End Portion 120>

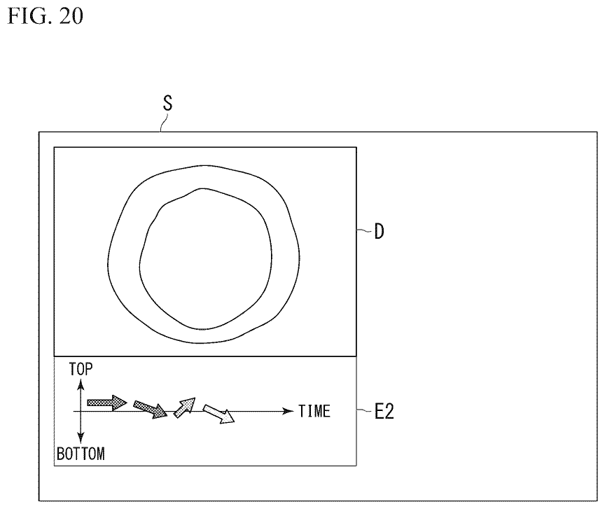

FIG. 20 is an explanatory diagram of an example in which information about the orientation of the distal end portion 120 is used as the second auxiliary information E2. An incorrect manipulation on the endoscope 100 may cause the distal end portion 120 to face in an unintended direction and cause the region of attention to disappear from the captured image D. In this case, if there is a history of the direction in which the distal end portion 120 faces, the practitioner can easily return the orientation of the distal end portion 120 to the orientation of the distal end portion 120 before the region of attention disappeared. Therefore, as shown in FIG. 20, the second auxiliary information E2 desirably includes a history of the orientation of the distal end portion 120. The history of the orientation of the distal end portion 120 shown in FIG. 20 is the history of the orientation of the distal end portion 120 in the up-down direction. The second auxiliary information E2 may include a history of the orientation of the distal end portion 120 in the left-right direction.

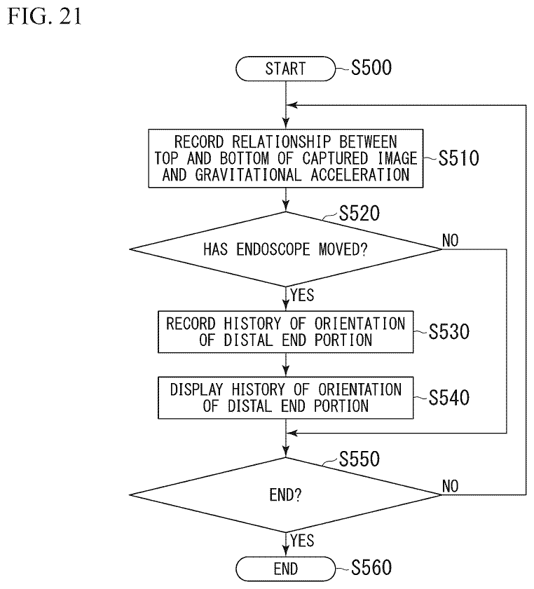

FIG. 21 is a control flowchart for displaying the history of the orientation of the distal end portion 120.

In step S510, the auxiliary information generation unit 270 records a relationship between the up-down direction of the captured image D and the output of the sensor 170 (gravitational acceleration). In step S520, the auxiliary information generation unit 270 determines whether the distal end portion 120 of the endoscope 100 has moved on the basis of the captured image D or the output of the sensor 170. In step S530, the auxiliary information generation unit 270 records the history of the orientation of the distal end portion 120. In step S550, the image synthesis unit 290 generates a synthetic image S including the captured image D and the second auxiliary information E2 generated by the auxiliary information generation unit 270, and outputs the synthetic image S to the display device 400. The display device 400 displays the received synthetic image S.

In addition, the auxiliary information generation unit 270 may generate second auxiliary information and add the second auxiliary information to the auxiliary information inferred by the inference model 281, even if the auxiliary information can be inferred by the inference model 281.

The endoscope system 500 according to the present embodiment can present auxiliary information (a cause and a countermeasure) for the loss of sight of a region of attention such as a lesion (a first region of attention) or a progress path (a second region of attention). The endoscope system 500 detects a change in the relative position between the distal end portion 120 of the endoscope 100 and the target part of the imaging target, and generates auxiliary information for the loss of sight of the region of attention according to different policies according to whether the change in the relative position is primarily based on the movement of the endoscope 100 or whether the change in the relative position is primarily based on the movement of the living body. The endoscope system 500 can generate second auxiliary information, even if the auxiliary information cannot be inferred by the inference model 281.

Although an embodiment of the present disclosure has been described above in detail with reference to the drawings, the specific configuration is not limited to this embodiment, and design modifications within the scope of the present disclosure are also included. Moreover, constituent elements shown in the above embodiment and the modified examples shown below can be configured in an appropriate combination.

In the above-described embodiment, the endoscope auxiliary information generation device performs diagnostic assistance with respect to images from the medical endoscope. However, a diagnosis target of the endoscope auxiliary information generation device is not limited to images from the medical endoscope. The endoscope auxiliary information generation device may perform diagnostic assistance with respect to captured images acquired from other imaging devices such as cameras, video cameras, industrial endoscopes, microscopes, robots with image acquisition functions, smartphones, portable phones, smart watches, tablet terminals, mobile devices of notebook PCs and the like.

The present disclosure can be applied to endoscope systems, etc.

[Aspect 1] An endoscope auxiliary information generation device comprising:
an image acquisition unit configured to sequentially acquire a plurality of images of an inside of a living body obtained by an imaging unit of an endoscope that images an inside of a lumen of a subject:
a region-of-attention detection unit configured to detect a region of attention having a specific feature in the image;
a relative position change detection unit configured to detect a change in a relative position between the imaging unit and a target object imaged by the imaging unit on the basis of the image; and
an auxiliary information generation unit configured to generate auxiliary information for guiding the endoscope to be manipulated in accordance with consecutive changes in the relative position.
   [Aspect 2] The endoscope auxiliary information generation device according to Aspect 1, wherein the auxiliary information generation unit generates the auxiliary information in accordance with a change in the region of attention when the relative position change detection unit determines that consecutive changes in the relative position are not changes in an image feature of a tissue in the lumen in a regular direction and detects that the change is primarily based on movement of the living body.
   [Aspect 3] The endoscope auxiliary information generation device according to Aspect 1, wherein the auxiliary information generation unit generates auxiliary information for supporting a path of the endoscope in a progress direction within the living body when the relative position change detection unit determines that consecutive changes in the relative position are changes in an image feature of a tissue in the lumen in an approximately regular direction and detects that the change is primarily based on movement of the living body.
   [Aspect 4] The endoscope auxiliary information generation device according to Aspect 3, wherein the auxiliary information for supporting the path in the travel direction is generated in accordance with information for identifying the lumen, and anatomical information in a lumen bending direction generated according to insertion into the identified lumen on the basis of an up-down direction of a screen when the endoscope is inserted and an examination posture of the subject.
   [Aspect 5] The endoscope auxiliary information generation device according to any one of Aspects 1 to 4, wherein the auxiliary information includes either a cause of loss of sight of the region of attention or a countermeasure for the loss of sight of the region of attention.
   [Aspect 6] The endoscope auxiliary information generation device according to Aspect 1,
wherein the auxiliary information generation unit further includes an inference model,
wherein the inference model is a model that identifies image frames before and after disappearance of the region of attention from the plurality of images obtained by the image acquisition unit and is trained using training data including annotations related to a cause of the disappearance for the image frame after the disappearance of the region of attention and is a model trained to infer the cause of the disappearance when the image frame in which the region of attention has disappeared is input, and
wherein the auxiliary information generation unit infers the cause of the disappearance for the image in which the region of attention has disappeared as the auxiliary information.
   [Aspect 7] The endoscope auxiliary information generation device according to Aspect 6,
wherein the inference model is a model that identifies image frames before and after disappearance of the specific feature from the images obtained by the image acquisition unit and is trained using training data further including annotations related to the countermeasure to recover from the disappearance for the image frame after the disappearance of the region of attention and is a model trained to infer the countermeasure when the image frame in which the region of attention has disappeared is input, and
wherein the auxiliary information generation unit further infers the countermeasure for the image in which the region of attention has disappeared as the auxiliary information.
   [Aspect 8] The endoscope auxiliary information generation device according to Aspect 1, wherein the auxiliary information includes the image before the region of attention disappears.
   [Aspect 9] The endoscope auxiliary information generation device according to Aspect 1, wherein the auxiliary information includes an orientation of a body of a patient.
   [Aspect 10] The endoscope auxiliary information generation device according to Aspect 1, wherein the auxiliary information includes a history of an orientation of the imaging unit of the endoscope.
   [Aspect 11] The endoscope auxiliary information generation device according to Aspect 6 or 7, wherein, when the auxiliary information cannot be inferred by the inference model, the auxiliary information generation unit generates any one of the image before the region of attention disappears, the orientation of the body of the patient, and the history of the orientation of the imaging unit of the endoscope as the auxiliary information.
   [Aspect 12] An endoscope auxiliary information generation method comprising:
sequentially acquiring a plurality of images of an inside of a living body obtained by an imaging unit of an endoscope:
detecting a region of attention having a specific feature in the image;
detecting a change in a relative position between the imaging unit and a target object imaged by the imaging unit on the basis of the image; and
generating auxiliary information for guiding the endoscope to be manipulated in accordance with consecutive changes in the relative position.
   [Aspect 13] The endoscope auxiliary information generation method according to Aspect 12, comprising generating the auxiliary information in accordance with the change in the region of attention when it is detected that the consecutive changes in the relative position are primarily based on movement of the living body.
   [Aspect 14] The endoscope auxiliary information generation method according to Aspect 12, comprising generating the auxiliary information for supporting a path of a progress direction within the living body of the endoscope when it is detected that the consecutive changes in the relative position are primarily based on movement of the endoscope.
   [Aspect 15] An endoscope auxiliary information generation program for causing a computer to:
sequentially acquire a plurality of images of an inside of a living body obtained by an imaging unit of an endoscope:
detect a region of attention having a specific feature in the image;
detect a change in a relative position between the imaging unit and a target object imaged by the imaging unit on the basis of the image; and
generate auxiliary information for guiding the endoscope to be manipulated in accordance with consecutive changes in the relative position.
   [Aspect 16] An inference model training method comprising:
detecting a region of attention having a specific feature in an in-image region from consecutive images obtained by an image acquisition unit using an endoscope for observing an inside of a living body; and
identifying image frames before and after the specific feature disappears from the images obtained by the image acquisition unit, annotating a cause of the disappearance and a countermeasure for the frame after the disappearance to create training data, and inferring and outputting the cause of the disappearance or the countermeasure when an image in which the region of attention having the specific feature has disappeared from an endoscopic image is input.
   [Aspect 17] An endoscope auxiliary information generation method in an insertion guide method when an endoscope is inserted from an end of a lumen of a specific internal organ of a subject, the endoscope auxiliary information generation method comprising steps of:
sequentially acquiring image information from an imaging unit provided on a distal end of the endoscope;
sequentially displaying the image information;
inputting posture information about a subject during an examination;
acquiring information about a lumen bending direction within the lumen of the specific internal organ; and
generating endoscope auxiliary information in accordance with anatomical information in a lumen bending direction generated according to insertion into the lumen of the specific internal organ on the basis of an up-down direction of the screen when the endoscope is inserted.
   [Aspect 18] An endoscope auxiliary information generation program comprising:
sequentially acquiring, by a computer, image information from an imaging unit provided on a distal end of an endoscope;
sequentially displaying, by the computer, the image information;
inputting, by the computer, posture information about a subject during an examination using the endoscope; and
generating, by the computer, endoscope auxiliary information in accordance with information about a lumen bending direction within a lumen of a specific internal organ and anatomical information in the lumen bending direction generated according to insertion into the lumen of the specific internal organ on the basis of an up-down direction of a screen when the endoscope is inserted.
   [Aspect 19] An endoscopic image processing device comprising:
an image acquisition unit configured to sequentially acquire image information from an imaging unit provided on a distal end of an endoscope;
a display control unit configured to sequentially display the image information; and
an auxiliary information generation unit configured to generate endoscope auxiliary information in accordance with information from an input unit configured to input posture information of a subject during an examination using the endoscope, information about a lumen bending direction within a lumen of a specific internal organ, and anatomical information in the lumen bending direction generated according to insertion into the lumen of the specific internal organ on the basis of an up-down direction of a screen when the endoscope is inserted.
   [Aspect 20] An endoscope auxiliary system comprising:
an image acquisition unit configured to sequentially acquire image information from an imaging unit provided on a distal end of an endoscope;
a display control unit configured to sequentially display the image information;
an input unit configured to input posture information about a subject during an examination using the endoscope; and
an auxiliary information generation unit configured to generate endoscope auxiliary information in accordance with information about a lumen bending direction within a lumen of a specific internal organ, the posture information, and anatomical information in the lumen bending direction generated according to insertion into the lumen of the specific internal organ on the basis of an up-down direction of a screen when the endoscope is inserted.

What is claimed is:

1. An endoscope auxiliary information generation device comprising:
an image acquisition unit configured to sequentially acquire a plurality of images of an inside of a living body obtained by an imaging unit of an endoscope that images an inside of a lumen of a subject:
a region-of-attention detection unit configured to detect a region of attention having a specific feature in the image;
a relative position change detection unit configured to detect a change in a relative position between the imaging unit and a target object imaged by the imaging unit on the basis of the image; and
an auxiliary information generation unit configured to generate auxiliary information for guiding the endoscope to be manipulated in accordance with consecutive changes in the relative position,
wherein the auxiliary information generation unit generates the auxiliary information in accordance with a change in the region of attention when the relative position change detection unit determines that consecutive changes in the relative position are not changes in an image feature of a tissue in the lumen in a regular direction and detects that the change is primarily based on movement of the living body.

2. The endoscope auxiliary information generation device according to claim 1, wherein the auxiliary information generation unit generates auxiliary information for supporting a path of the endoscope in a progress direction within the living body when the relative position change detection unit determines that consecutive changes in the relative position are changes in an image feature of a tissue in the lumen in an approximately regular direction and detects that the change is primarily based on movement of the living body.

3. The endoscope auxiliary information generation device according to claim 2, wherein the auxiliary information for supporting the path in the travel direction is generated in accordance with information for identifying the lumen, and anatomical information in a lumen bending direction generated according to insertion into the identified lumen on the basis of an up-down direction of a screen when the endoscope is inserted and an examination posture of the subject.

4. The endoscope auxiliary information generation device according to claim 1, wherein the auxiliary information includes either a cause of loss of sight of the region of attention or a countermeasure for the loss of sight of the region of attention.

5. The endoscope auxiliary information generation device according to claim 1,
wherein the auxiliary information generation unit further includes an inference model,
wherein the inference model is a model that identifies image frames before and after disappearance of the region of attention from the plurality of images obtained by the image acquisition unit and is trained using training data including annotations related to a cause of the disappearance for the image frame after the disappearance of the region of attention and is a model trained to infer the cause of the disappearance when the image frame in which the region of attention has disappeared is input, and
wherein the auxiliary information generation unit infers the cause of the disappearance for the image in which the region of attention has disappeared as the auxiliary information.

6. The endoscope auxiliary information generation device according to claim 5,
wherein the inference model is a model that identifies image frames before and after disappearance of the specific feature from the images obtained by the image acquisition unit and is trained using training data further including annotations related to the countermeasure to recover from the disappearance for the image frame after the disappearance of the region of attention and is a model trained to infer the countermeasure when the image frame in which the region of attention has disappeared is input, and
wherein the auxiliary information generation unit further infers the countermeasure for the image in which the region of attention has disappeared as the auxiliary information.

7. The endoscope auxiliary information generation device according to claim 1, wherein the auxiliary information includes the image before the region of attention disappears.

8. The endoscope auxiliary information generation device according to claim 1, wherein the auxiliary information includes an orientation of a body of a patient.

9. The endoscope auxiliary information generation device according to claim 1, wherein the auxiliary information includes a history of an orientation of the imaging unit of the endoscope.

10. The endoscope auxiliary information generation device according to claim 5, wherein, when the auxiliary information cannot be inferred by the inference model, the auxiliary information generation unit generates any one of the image before the region of attention disappears, the orientation of the body of the patient, and the history of the orientation of the imaging unit of the endoscope as the auxiliary information.

11. An inference model training method comprising:
detecting a region of attention having a specific feature in an in-image region from consecutive images obtained by an image acquisition unit using an endoscope for observing an inside of a living body; and
identifying image frames before and after the specific feature disappears from the images obtained by the image acquisition unit, annotating a cause of the disappearance and a countermeasure for the frame after the disappearance to create training data, and inferring and outputting the cause of the disappearance or the countermeasure when an image in which the region of attention having the specific feature has disappeared from an endoscopic image is input.

12. An endoscope auxiliary information generation method in an insertion guide method when an endoscope is inserted from an end of a lumen of a specific internal organ of a subject, the endoscope auxiliary information generation method comprising steps of:
sequentially acquiring image information from an imaging unit provided on a distal end of the endoscope;
sequentially displaying the image information;
inputting posture information about a subject during an examination;
acquiring information about a lumen bending direction within the lumen of the specific internal organ; and
generating endoscope auxiliary information in accordance with anatomical information in a lumen bending direction generated according to insertion into the lumen of the specific internal organ on the basis of an up-down direction of the screen when the endoscope is inserted.
